(19) United States
(12) Patent Application Publication
(10) Pub. No.: US 2022/0054379 A1
(43) Pub. Date: Feb. 24, 2022

(54) LOW-ODOR THIOGLYCOLIC ACID COMPOSITION

(71) Applicant: Arkema France, Colombes (FR)

(72) Inventors: Bernard Monguillon, Colombes (FR); Pierre-Thomas Skowron, Lacq (FR)

(73) Assignee: Arkema France, Colombes (FR)

(21) Appl. No.: 17/415,531

(22) PCT Filed: Dec. 18, 2019
(86) PCT No.: PCT/FR2019/053145

§ 371 Date: Jun. 17, 2021

(30) Foreign Application Priority Data
Dec. 21, 2018 (FR) 1873828

(51) Int. Cl. A61K 8/365; A61K 8/46; A61K 8/67; A61Q 5/02; A61Q 5/04; A61Q 9/04

(52) U.S. Cl. CPC A61K 8/365; A61K 8/46; A61K 8/676; A61K 2800/882; A61Q 5/04; A61Q 9/04; A61Q 5/02

(57) ABSTRACT

The invention relates to a low-odor thioglycolic acid composition comprising a linear or cyclic organic acid comprising from 1 to 3 carboxylic acid functions and a saturated or unsaturated, linear or branched or cyclic hydrocarbon chain containing from 1 to 10 carbon atoms; and also to the process for preparing the composition and to its uses.

### CROSS-REFERENCE TO RELATED APPLICATIONS

This is the national phase of International Application No. PCT/FR2019/053145, filed 18 Dec. 2019, which claims priority to French Application No. FR 1873828, filed 21 Dec. 2018. The disclosure of each of these applications is incorporated herein by reference in its entirety for all purposes.

### FIELD OF THE INVENTION

The present invention relates to a low-odor thioglycolic acid (hereinafter referred to as “TGA”) composition, to the process for preparing same and to the uses of said composition.

### BACKGROUND OF THE INVENTION

Thioglycolic acid, of the following formula:

HS—CH₂—COCH

and also its salts and esters, is used in numerous fields such as cosmetics (for example in shampoos, hair curling or relaxing compositions and hair-removal creams), leather treatment, cleaning solutions or else in the preparation of polymers as chain-transfer agent. However, the use of thioglycolic acid has a drawback due to its unpleasant odor, notably distinguished by a sulfurous and acidic, even rancid, odor combined with a scorched and/or burnt odor. Moreover, under certain conditions, thioglycolic acid can decompose into hydrogen sulfide (H₂S) and other compounds such as highly malodorous light mercaptans.

This odor is disturbing when handling the TGA and can end up in the finished products containing it, which represents a major drawback with its use, in particular in the cosmetics field.

In order to counter this unpleasant odor, a number of strategies have been employed by manufacturers: masking the odor of the TGA using odor-masking agents, or reducing the release of H₂S and/or of light mercaptans which may be responsible for the odor.

Mention may thus be made, for example, of the use of:
odor-masking agents of natural substance, natural substance extract or fragrancing base type;
adsorbents such as charcoal or zeolites or cyclodextrins which make it possible to reduce the amount of H₂S and/or of light mercaptans released; or
specific additives likewise making it possible to reduce the amount of H₂S and/or of light mercaptans released, such as polyphenols or metal oxides.

Thus, the majority of the solutions proposed are solely concerned with the masking or the adsorption of malodorous compounds. These solutions do not take into account the fact that the thioglycolic acid obtained industrially contains impurities, some of which may be partly responsible for the unpleasant odor of the TGA.

There is therefore a need for a low-odor thioglycolic acid composition. More particularly, there is a need for a low-odor thioglycolic acid composition which can be obtained industrially. There is also a need for a thioglycolic acid composition the unpleasant odor of which is masked or reduced, indeed even eliminated, in particular in the finished products containing said composition.

### SUMMARY OF THE INVENTION

An object of the invention is therefore to propose low-odor compositions comprising thioglycolic acid. More particularly, an object of the present invention is to propose low-odor compositions comprising a thioglycolic acid obtained by an industrial preparation process, more particularly by an industrial preparation process using monochloroacetic acid (MCAA) or its salts.

An object of the present invention is also to propose a low-odor thioglycolic acid composition useful in particular in the cosmetics field, leather treatment, cleaning solutions or else in the preparation of polymers as chain-transfer agent.

The present invention thus relates to a composition C comprising:
at least one linear or cyclic organic acid A comprising from 1 to 3 carboxylic acid functions and comprising a saturated or unsaturated, linear or branched or cyclic hydrocarbon chain containing from 1 to 10 carbon atoms;
said carbon atoms being optionally substituted by one or more substituents chosen from —OH and ═O; or one of its salts;
a composition B comprising:
   a) thioglycolic acid, or one of its salts or esters; and
   b) isopropyl thioglycolate (IPTG) in an amount of between strictly greater than 0 and 130 ppm, preferably between 5 and 80 ppm, and more preferentially between 30 and 70 ppm, for example between 20 and 70 ppm; and
optionally a solvent S;
   said organic acid(s) A and thioglycolic acid being in proportions such that the (organic acid(s) A/thioglycolic acid) molar ratio is between 0.0004 and 0.1, preferably between 0.0004 and 0.004.

### DETAILED DESCRIPTION OF THE INVENTION

In contrast to the known solutions from the prior art as mentioned above, the present inventors have discovered that the combination of at least one organic acid having 1, 2 or 3 —C(O)OH functions with TGA obtained by an industrial process made it possible to mask, reduce and even eliminate the odor of said TGA. More particularly, the composition C according to the invention has a less intense odor and a greater hedonic nature than a thioglycolic acid composition B as defined above. Thus, the sulfurous and/or scorched and/or burnt odor of TGA composition B is in particular masked, reduced or even eliminated by virtue of the addition of at least one organic acid as defined above. In particular, the sulfurous odor of the TGA composition B is masked, reduced or even eliminated by virtue of the addition of at least one organic acid A as defined above.

According to one embodiment, such a composition B corresponds to a thioglycolic acid composition obtained industrially, for example using MCAA or one of its salts, according to the process described below.

The industrial preparation of thioglycolic acid is described in the publication “Thioglycolic acid”, Y. Labat, Kirk-Othmer Encyclopedia of Chemical Technology, Fourth Edition, vol. 24, 1997, ISBN 0-471-52693-2, in particular on pages 3-6 “Manufacturing, Processing, and Storage”). Thioglycolic acid is produced by the reaction of monochloroacetic acid (MCAA) or one of its salts with alkali metal hydrosulfides such as NaSH or NH₄SH in aqueous medium. The reaction mixture is then acidified so as to liberate thioglycolic acid. These two reaction steps are summarized in the following equations:

Cl—CH₂—COOH+2XSH→HS—CH₂—COO⁻X⁺+XCI+H₂S WITH X═NH₄ OR Na (1)

HS—CH₂—COO⁻X⁺+HCI→HS—CH₂—COOH+XCI (2)

The thioglycolic acid obtained is then extracted from the aqueous solution with an organic solvent, preferentially of ether type such as diisopropyl ether. The organic solvent is evaporated and then the thioglycolic acid is purified by vacuum distillation.

The thioglycolic acid may then undergo topping to remove light compounds. Finally, the thioglycolic acid may undergo tailing in order to remove heavy compounds.

Diisopropyl ether (DIPE or IPE) is an extraction solvent commonly used in industry. In the process for producing TGA, IPE is in contact with hydrochloric acid and can undergo acidic cleavage to form isopropanol. Esterification of the isopropanol with the TGA forms isopropyl thioglycolate (IPTG).

At the end of this industrial manufacturing process, the thioglycolic acid-based composition obtained may thus comprise IPE and IPTG.

### Definitions

The term “low-odor thioglycolic acid composition” is understood in particular to mean a composition C as according to the invention. In particular, said composition C has an odor exhibiting a lower or equal intensity and a greater or equal hedonic nature with respect to a thioglycolic acid composition having a strong odor as defined below.

The term “thioglycolic acid composition having a strong odor” is understood in particular to mean a composition B as according to the invention. In particular, said thioglycolic acid composition B is obtained following an industrial process using MCAA as starting product.

In particular, the composition C according to the invention exhibits a sulfurous odor (or sulfurous note) which is masked, reduced or eliminated compared to a thioglycolic acid composition B not comprising any organic acid A as defined above.

The term “odor” is understood to mean the olfactory perception of a product, here the compositions B and C, which can be characterized by sensory analysis. The intensity, the hedonic nature and the description of the odor are parameters typically employed in the sensory characterization of an odor. The determination of the concentration of an odor by dynamic olfactometry can be performed in accordance with the standard EN 13725 (October 2003).

The term “olfactory intensity” is understood for example to mean the measurement of intensity on the basis of the German standard VDI 3882 part 1. The panel of selected people can be in accordance with the standard EN 13725. The intensity can be evaluated on a scale from 0 to 5 (5 very strong, 4 strong, 3 medium, 2 weak, 1 very weak, 0 not detectable). According to one embodiment, the compositions according to the invention exhibit an average intensity of between 2 and 4, around 3, for example between 2.5 and 3.5.

The term “hedonic nature” is understood to mean the pleasant or unpleasant nature of an odor. For example, the measurement of the hedonic nature can be based on the German standard VDI 3882 part 2. The panel of selected people can be in accordance with the standard EN 13725. The hedonic nature can be evaluated on a scale from −4 to +4 (+4 extremely pleasant, +3 very pleasant, +2 pleasant, +1 faintly pleasant, 0 neutral, −1 faintly unpleasant, −2 unpleasant, −3 very unpleasant and −4 extremely unpleasant). According to one embodiment, the hedonic nature of the compositions according to the invention is between −1 and +1, for example between −0.3 and +1.5.

The term “carboxylic acid function” is understood to mean a —C(O)OH group. The term “cyclic organic acid A” is understood to mean an organic acid the carboxylic acid function of which is cyclized, for example ascorbic acid.

The term “(C₁-C₁₀)-alkyl” denotes saturated aliphatic hydrocarbons which may be linear or branched and comprise from 1 to 10 carbon atoms. Preferably, said alkyls comprise from 1 to 5 carbon atoms, or even from 1 to 4 carbon atoms. The term “branched” is understood to mean that an alkyl group is substituted along the main alkyl chain. The alkyls preferred according to the invention are methyl, ethyl, propyl and butyl.

The term “(C₁-C₁₀)-alkenyl” refers to a (C₁-C₁₀)-alkyl comprising at least one double bond, preferably just one double bond.

The term “(C₃-C₁₀)-cycloalkyl” denotes cyclic saturated aliphatic hydrocarbons comprising from 3 to 10 carbon atoms, in particular cyclopropyl or cyclohexyl.

The term “(C₆-C₁₀)-aryl” denotes monocyclic, bicyclic or tricyclic aromatic hydrocarbons, in particular phenyl and naphthyl.

The salts of organic acid A and the salts and esters of thioglycolic acid are known to those skilled in the art. The esters of TGA are for example the methyl, ethyl, glycerol or sorbitol esters. For example, for thioglycolic acid, it is possible to envisage the Na, Ca, K, NH₄ and monoethanolamine salts. According to one embodiment, the organic acids A are chosen from the compounds of the following formula (I′):

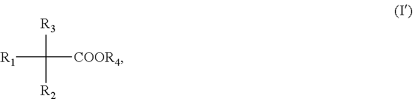

in which R₁, R₂ and R₃ are as defined for formula (I) and R₄ is chosen from the group consisting of H, Na, Ca, K and NH₄.

According to a preferred embodiment, said composition C does not comprise any salts of organic acid A or any salts of thioglycolic acid.

The term “ppm” is understood to mean parts per million, by expressing a mass fraction (1 ppm=1 mg/kg).

### Compositions C

A subject of the present invention is thioglycolic acid compositions C as defined above, and preferably having a low odor.

According to one embodiment, said organic acid(s) A and thioglycolic acid are in proportions such that the (organic acid(s) A/thioglycolic acid) molar ratio is between 0.0005 and 0.005, preferably between 0.0007 and 0.008.

According to one embodiment, the (organic acid(s) A/thioglycolic acid) molar ratio is between 0.0008 and 0.008.

According to one embodiment, said composition C comprises between 0.1% and 99.9%, preferably between 50% and 99.9% by weight of thioglycolic acid relative to the total weight of composition C. More preferentially, composition C according to the invention comprises about 70%, 80%, 85%, 97%, 98%, 99% or 99.9%, preferably 80% or 99% by weight of thioglycolic acid relative to the total weight of said composition C. For example, composition C comprises at least 99% by weight of thioglycolic acid relative to the total weight of said composition C.

### Organic Acid A

According to one embodiment, composition C comprises an organic acid A.

According to one embodiment, composition C comprises at least one linear or cyclic organic acid A consisting of 1 to 3 carboxylic acid functions and a saturated or unsaturated, linear or branched or cyclic hydrocarbon chain containing from 1 to 10 carbon atoms;

said carbon atoms being optionally substituted by one or more substituents chosen from —OH and ═O; or one of its salts.

According to one embodiment, composition C comprises at least one linear organic acid A comprising 1 carboxylic acid function and comprising a saturated, linear, branched or cyclic hydrocarbon chain containing from 1 to 10 carbon atoms; said carbon atoms being optionally substituted by one or more substituents chosen from —OH and ═O; or one of its salts.

According to one embodiment, said at least one organic acid A is ascorbic acid or an organic acid of general formula (I) below:

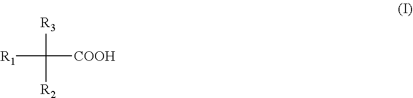

in which:
R₁ is chosen from the group consisting of:
   —H, —OH, (C₁-C₁₀)-alkyl and (C₂-C₁₀)-alkenyl; said (C₁-C₁₀)-alkyl and (C₂-C₁₀)-alkenyl being optionally substituted by at least one substituent chosen from the group consisting of: —OH, —C(O)OH and ═O;
R₂ is —H, —OH or (C₁-C₁₀)-alkyl;
R₃ is H;
   or R₂ and R₃ form, together with the carbon atom to which they are attached, a C═O group;
   or R₁ and R₂ form, together with the carbon atom to which they are attached, a (C₃-C₁₀)-cycloalkyl or (C₆-C₁₀)-aryl group,
   said (C₃-C₁₀)-cycloalkyl and (C₆-C₁₀)-aryl groups being optionally substituted by at least one substituent chosen from the group consisting of:
   —OH, —C(O)OH and ═O;
      or one of its salts.

According to one embodiment, R₁ is chosen from the group consisting of: —H, —OH and (C₁-C₈)-alkyl; said (C₁-C₈)-alkyls being optionally substituted by one, two, three or four substituents chosen from the group consisting of: —OH, —C(O)OH and ═O.

According to one embodiment, R₂ is —H or —OH. According to one embodiment, R₂ and R₃ are H.

According to a preferred embodiment, R₁ is a (C₁-C₁₀)-alkyl optionally substituted by at least one substituent chosen from the group consisting of: —OH, —C(O)OH and ═O and R₃ is H.

According to one embodiment, said at least one organic acid A is an organic acid of general formula (I) as defined above.

According to one embodiment, the organic acid A is chosen from the group consisting of acetic acid, glycolic acid, lactic acid, propionic acid, citric acid, salicylic acid, cyclohexanecarboxylic acid, 2-ethylbutyric acid, pyruvic acid, levulinic acid, ascorbic acid, acetonedicarboxylic acid, 2-keto-L-gluconic acid, and mixtures thereof.

According to one embodiment, the organic acid A is chosen from the group consisting of acetic acid, glycolic acid, lactic acid, propionic acid, citric acid, salicylic acid, cyclohexanecarboxylic acid, 2-ethylbutyric acid, pyruvic acid, levulinic acid, acetonedicarboxylic acid, 2-keto-L-gluconic acid, and mixtures thereof.

According to one embodiment, the organic acid A is chosen from the group consisting of acetic acid, glycolic acid, lactic acid, propionic acid, citric acid, salicylic acid, cyclohexanecarboxylic acid, 2-ethylbutyric acid, pyruvic acid, levulinic acid, ascorbic acid, acetonedicarboxylic acid, and mixtures thereof.

According to one embodiment, the organic acid A is chosen from the group consisting of acetic acid, propionic acid, 2-ethylbutyric acid, pyruvic acid, ascorbic acid, acetonedicarboxylic acid, keto-L-gluconic acid, citric acid, and mixtures thereof.

According to one embodiment, the organic acid A is chosen from the group consisting of acetic acid, propionic acid, 2-ethylbutyric acid, pyruvic acid, acetonedicarboxylic acid, and mixtures thereof.

Preferentially, the organic acid A is chosen from acetic acid and 2-ethylbutyric acid, preferably acetic acid.

According to another embodiment, the organic acid A is neither ascorbic acid nor 2-keto-L-gluconic acid, or their salts. According to another embodiment, the organic acid A is neither ascorbic acid nor 2-keto-L-gluconic acid nor erythorbic acid, or their salts.

According to one embodiment, composition C does not comprise any odor-masking agents other than the organic acid A as defined above. For example, composition C does not comprise any fragrancing base, for example of natural substances or of extracts of natural substances.

### Composition B

Composition B is included in composition C according to the invention. Composition C can be considered as a composition B which has been additivated in order to mask, reduce or even eliminate its odor.

According to one embodiment, composition B comprises:

a) thioglycolic acid or one of its salts;

b) isopropyl thioglycolate (IPTG) in an amount of between strictly greater than 0 and 130 ppm, preferably between 5 and 80 ppm, and more preferentially between 30 and 70 ppm; and

c) diisopropyl ether (IPE) in an amount of between 5 and 100 ppm, preferably between 10 and 80 ppm.

According to one embodiment, composition B comprises:

a) from 0.1% to 99.9%, preferably from 50% to 99.9% by weight of thioglycolic acid, or one of its salts, relative to the total weight of said composition B;

b) isopropyl thioglycolate (IPTG) in an amount of between strictly greater than 0 and 130 ppm, preferably between 5 and 80 ppm, and more preferentially between 30 and 70 ppm; and

c) diisopropyl ether (IPE) in an amount of between 5 and 100 ppm, preferably between 10 and 80 ppm.

According to one embodiment, said composition B is obtained by a process comprising the following steps:

a) mixing monochloroacetic acid (MCAA) or one of its salts with NaSH or NH₄SH in an aqueous medium;

b) acidifying the reaction mixture obtained in step a) to obtain thioglycolic acid, said acidification preferably being performed by addition of hydrochloric acid;

c) extracting the thioglycolic acid obtained in step b) from the aqueous phase with diisopropyl ether;

d) evaporating the diisopropyl ether from the organic phase obtained in step c); and

e) purifying, preferably by vacuum distillation, the thioglycolic acid.

### Solvent S and Other Additives

According to one embodiment, said composition C comprises water or an alcohol as solvent. Composition C is generally an aqueous formulation which may be prepared in the form of a concentrated mixture which is diluted by the end user. As a variant, composition C may also be a ready-to-use formulation, that is to say one which does not need to be diluted.

Composition C may optionally comprise one or more additives, for example rheological or texturing agents, thickeners, surfactants, foaming agents, antifoams, and others known to those skilled in the art.

### Uses According to the Invention

A subject of the present invention is also the use of an organic acid A as defined above for masking, reducing or eliminating the odor of a composition B as defined above. Organic acid A can be considered to be an odor-masking agent for composition B.

According to one embodiment, said use makes it possible to mask, reduce or eliminate the sulfurous and/or scorched and/or burnt odor of said composition B as defined above.

According to one embodiment, said use according to the invention makes it possible to reduce the intensity of the odor and/or to enhance the hedonic nature of the odor of said composition B as defined above. According to one embodiment, said use according to the invention makes it possible to obtain a weaker intensity of the odor and/or a greater hedonic nature of the composition C, compared to composition B as defined above.

The present invention also relates to the use of a composition C according to the invention in the cosmetics field (for example in shampoos, hair curling or relaxing compositions and hair-removal creams), leather treatment, cleaning solutions or else in the preparation of polymers as chain-transfer agent.

The present invention also relates to a kit comprising a composition B as defined above and an organic acid A as defined above.

### Process for Preparing the Composition C

A subject of the present invention is a process for preparing a composition C as defined above, characterized in that it comprises a step of mixing at least one organic acid A as defined above and a composition B as defined above. The preparation of such a composition C may be effected in a manner known to those skilled in the art, by simple mixing of composition B with at least one organic acid A as defined above.

According to one embodiment, said composition C comprises:
at least one linear or cyclic organic acid A comprising from 1 to 3 carboxylic acid functions and comprising a saturated or unsaturated, linear or branched or cyclic hydrocarbon chain containing from 1 to 10 carbon atoms;
said carbon atoms being optionally substituted by one or more substituents chosen from —OH and ═O; or one of its salts;
thioglycolic acid (TGA), or one of its salts or esters; and
isopropyl thioglycolate (IPTG) in an amount of between strictly greater than 0 and 130 ppm, preferably between 5 and 80 ppm, and more preferentially between 30 and 70 ppm relative to the amount of TGA; and
optionally a solvent S;
   said organic acid(s) A and thioglycolic acid being in proportions such that the (organic acid(s) A/thioglycolic acid) molar ratio is between 0.0004 and 0.1, preferably between 0.0004 and 0.004.

### EXAMPLES

### Example 1

### Olfactory Tests Performed on the Thioglycolic Acid Compositions

### I. Sample Preparation:

A composition B is prepared according to the following process:

Monochloroacetic acid (1 equivalent) is reacted with ammonium hydrosulfide (2 equivalents) in a tubular reactor at a temperature of 15 to 100° C. under a pressure of 5 to 25 bar of hydrogen sulfide. The product obtained is then acidified with 33% hydrochloric acid until an acidic pH is obtained, and then the thioglycolic acid is extracted with diisopropyl ether (IPE). The IPE is then evaporated and the TGA is then distilled.

The composition B obtained has the following characteristics:

| Compound | Unit | Amount |
|---|---|---|
| TGA | % | 99.3 |
| IPE | ppm | 17 |
| MCAA | ppm | <0.05 |
| IPTG | ppm | 60 |

Next, 100 g of this composition B was mixed with the following organic acids A and 15 mL of each of the solutions obtained was subjected to olfactory analysis. These compositions correspond to the compositions C according to the invention.

| | Organic acid A added | | A/TGA |
|---|---|---|---|
| Sample | Amount | | molar |
| number | (ppm) | Type | ratio |
| 1 | — | — | |
| 2 | 500 | acetic | 0.0008 |
| 5 | 2000 | acetic | 0.0031 |
| 6 | 1000 | glycolic | 0.0012 |
| 7 | 1000 | lactic | 0.0010 |
| 8 | 1000 | propionic | 0.0012 |
| 9 | 1000 | citric | 0.0005 |
| 10 | 1000 | salicylic | 0.0007 |
| 11 | 1000 | cyclohexanecarboxylic | 0.0007 |
| 12 | 1000 | 2-ethylbutyric | 0.0008 |
| 13 | 1000 | pyruvic | 0.0010 |
| 14 | 1000 | levulinic | 0.0008 |
| 16 | 1000 | ascorbic | 0.0005 |
| 17 | 1000 | acetonedicarboxylic | 0.0006 |
| 18 | 1000 | 2-keto-L-gluconic | 0.0005 |

### II. Sampling Method and Analytical Method:

### Sampling:

For each sample, a volume of 0.1 μL was taken so as to obtain a gas phase concentration of 1 ppm, i.e. 4.5 mg·m⁻³. This volume was introduced into a Nalophan™ bag, and then a neutral gas (air) was added at a controlled flow rate (see EN 13725, § 6.2 and 6.4). The bags were then placed in an oven for 1 h at 25° C.

The concentrations introduced and the sampling parameters are as follows:

| | | | Inj. | | Concen- |
|---|---|---|---|---|---|
| | | Sampling | vol. in | | tration in |
| Samples | Temperature | time | μL | Density | mg · m⁻³ |
| All | 25° C. | 20 min | 0.1 | 1.33 | 4.5 |
| samples | | | | | |

### III. Sensory Analyses:

1. Performance of Sensory Analyses:

The sensory analyses were carried out on the same day of sampling by way of olfactometer and odor panelists as described by standard EN 13725 (§ 6). The flow rate is at least 20 l/min. The time allocated for evaluating the presented stimulus does not exceed 15 s. The interval of time between the stimuli must be sufficient to avoid adaptation of the panelists to the odor (§ 8 EN 13725).

After sensation, the panelists then indicated their perception according to the parameters and scales indicated below:

a) Intensity:

Measurements of intensity, based on a German standard (VDI 3882 part 1), were performed with a panel of 6 selected and trained people based on a reference scale of n-butanol in accordance with the standard EN 13725 (§ 6.4.2).

During the measurements, the odor panelists directly smell the sampled product and evaluate its intensity on a scale from 0 to 5.

| Odor intensity | Scale |
|---|---|
| Very strong | 5 |
| Strong | 4 |
| Medium | 3 |
| Weak | 2 |
| Very weak | 1 |
| Not detectable | 0 |

The average intensity of the composition is calculated according to the arithmetic mean of each individual result.

b) Hedonic Nature:

The measurements of the hedonic nature, based on a German standard (VDI 3882 part 2), were also performed with a panel of 6 selected people in accordance with the standard EN 13725.

For each sample, the panelists gave their perception of the pleasant or unpleasant nature of the odor according to a scale from −4 to +4.

| Hedonic nature | Scale |
|---|---|
| Extremely pleasant | +4 |
| Very pleasant | +3 |
| Pleasant | +2 |
| Faintly pleasant | +1 |
| Neutral | 0 |
| Faintly unpleasant | −1 |
| Unpleasant | −2 |
| Very unpleasant | −3 |
| Extremely unpleasant | −4 |

c) Odor Characterization:

The characterization of the odors is performed by expert odor panelists.

### IV. Results:

The table above gives the average results of the 6 panelists for the 18 samples tested, for intensity and hedonic nature, at 25° C.

| | | | Average |
|---|---|---|---|
| Sample | Type of organic | Average | hedonic |
| number | acid A | intensity | nature |
| 1 | — (TGA alone) | 3.33 | −0.33 |
| 2 | acetic | 2.83 | 0.17 |
| 5 | acetic | 3.33 | −0.17 |
| 6 | glycolic | 2.83 | −0.33 |
| 7 | lactic | 3.00 | −0.33 |
| 8 | propionic | 3.00 | 0.17 |
| 9 | citric | 2.67 | −0.17 |
| 10 | salicylic | 3.00 | −0.50 |
| 11 | cyclohexanecarboxylic | 3.17 | −0.67 |
| 12 | 2-ethylbutyric | 2.83 | 1.00 |
| 13 | pyruvic | 2.83 | 0.00 |
| 14 | levulinic | 2.83 | −0.50 |
| 16 | ascorbic | 3.17 | −0.17 |
| 17 | acetonedicarboxylic | 3.17 | 0.67 |
| 18 | 2-keto-L-gluconic | 4.17 | 0.33 |

1. Description of the Intensity and of the Hedonic Nature Obtained:

Sample 1 of TGA not treated with an organic acid A has an average intensity of 3.33 and a hedonic nature of −0.33.

It is observed that the addition of an organic acid A to a composition of TGA comprising 60 ppm of IPTG enables a lower intensity and/or a greater hedonic nature to be obtained compared to the same composition of TGA without the addition of organic acid A.

Intensities of between 2.83 and 4.17 and hedonic assessments of between −0.67 and 1 are observed.

Values lying around 0, in particular between −0.17 and 1, describe neutral, indeed even pleasant, odors.

The sample perceived as being the most intense (4.17, i.e. between strong and very strong) is sample 18. The least intense is sample 9, with 2.67 (weak-medium).

Regarding the hedonic nature, 9 samples out of the 18 are considered as being relatively neutral, with values of around 0 (from −0.17 to +0.17), and 3 samples as being slightly pleasant (samples 12, 17 and 18).

2. Sensory Profiles of All Samples:

Overall, the samples considered as being the most unpleasant (10, 11 and 14) were all described as having a significant sulfurous note.

The samples considered as being the most pleasant are characterized by a faint (sample 12) or absent (samples 17 and 18) sulfurous note and a relatively faint rancid/acidic note. The addition of an organic acid A according to the invention thus makes it possible to mask, reduce or even eliminate the sulfurous note of the TGA produced industrially. The scorched and/or burnt notes are also fainter when the TGA produced industrially is additivated in accordance with the invention.

1. A composition C comprising:
at least one linear or cyclic organic acid A comprising from 1 to 3 carboxylic acid functions and comprising a saturated or unsaturated, linear or branched or cyclic hydrocarbon chain containing from 1 to 10 carbon atoms;
the carbon atoms being optionally substituted by one or more substituents chosen from —OH and ═O; or one of its salts;
a composition B comprising:
a/ thioglycolic acid or one of its salts or esters; and
b/ isopropyl thioglycolate (IPTG) in an amount of between greater than 0 and 130 ppm; and
optionally a solvent S;
the organic acid(s) A and thioglycolic acid being in proportions such that the (organic acid(s) A/thioglycolic acid) molar ratio is between 0.0004 and 0.1.

2. The composition as claimed in claim 1, wherein the at least one organic acid A is ascorbic acid or an organic acid of the following general formula (I): 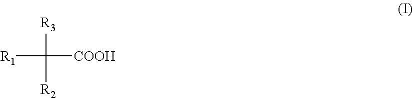
in which:
R₁ is chosen from the group consisting of: —H, —OH, (C₁-C₁₀)-alkyl and (C₂-C₁₀)-alkenyl;
the (C₁-C₁₀)-alkyl and (C₂-C₁₀)-alkenyl being optionally substituted by at least one substituent chosen from the group consisting of: —OH, —C(O)OH and ═O;
R₂ is —H, —OH or (C₁-C₁₀)-alkyl;
R₃ is H;
or R₂ and R₃ form, together with the carbon atom to which they are attached, a C═O group;
or R₁ and R₂ form, together with the carbon atom to which they are attached, a (C₃-C₁₀)-cycloalkyl or (C₆-C₁₀)-aryl group,
the (C₃-C₁₀)-cycloalkyl and (C₆-C₁₀)-aryl groups being optionally substituted by at least one substituent chosen from the group consisting of:
—OH, —C(O)OH and ═O;
or one of its salts.

3. The composition as claimed in claim 1, wherein the organic acid A is chosen from the group consisting of acetic acid, glycolic acid, lactic acid, propionic acid, citric acid, salicylic acid, cyclohexanecarboxylic acid, 2-ethylbutyric acid, pyruvic acid, levulinic acid, ascorbic acid, acetonedicarboxylic acid, 2-keto-L-gluconic acid, and mixtures thereof.

4. The composition as claimed in claim 1, wherein the organic acid A is chosen from acetic acid and 2-ethylbutyric acid.

5. The composition as claimed in claim 1, wherein the composition B comprises:
a/ thioglycolic acid or one of its salts;
b/ isopropyl thioglycolate (IPTG) in an amount of between greater than 0 and 130 ppm; and
c/ diisopropyl ether (IPE) in an amount of between 5 and 100 ppm.

6. The composition as claimed in claim 1, wherein the (organic acid(s) A/thioglycolic acid) molar ratio is between 0.0008 and 0.008.

7. The composition as claimed in claim 1, comprising between 50% and 99.9% by weight of thioglycolic acid relative to the total weight of the composition.

8. The composition as claimed in claim 1, wherein the composition B is obtained by a process comprising:
a/ mixing monochloroacetic acid (MCAA) or one of its salts with NaSH or NH₄SH in an aqueous medium;
b/ acidifying the reaction mixture obtained in step a) to obtain thioglycolic acid;
c/ extracting the thioglycolic acid obtained in step b) from the aqueous phase with diisopropyl ether;
d/ evaporating the diisopropyl ether from the organic phase obtained in step c); and
e/ purifying the thioglycolic acid.

9. A method for masking, reducing or eliminating the odor of a composition B, the method comprising contacting the composition B with at least one organic acid A as defined in claim 1, wherein composition B comprises:
a/ thioglycolic acid or one of its salts; and
b/ isopropyl thioglycolate (IPTG) in an amount of between strictly greater than 0 and 130 ppm.

10. The use of a composition C as claimed in claim 1 in the cosmetics field, leather treatment, cleaning solutions or in the preparation of polymers as chain-transfer agent.

11. A kit comprising a composition B and an organic A as defined in claim 1.

12. A process for preparing a composition C as claimed in claim 1, comprising mixing at least one of the organic acid A and a composition B comprising:
a/ thioglycolic acid or one of its salts; and
b/ isopropyl thioglycolate (IPTG) in an amount of between greater than 0 and 130 ppm.
